(19) United States
(12) Patent Application Publication
(10) Pub. No.: US 2023/0181776 A1
(43) Pub. Date: Jun. 15, 2023

(54) DEVICE AND METHOD FOR STEAM DISINFECTION OF PRODUCTS

(71) Applicants: Frieder LINDER, St. Leon-Rot (DE); Harald SAUER, Heidelberg (DE); Alexander Peter KRÜGER, Heidelberg (DE); Michael Markus HECKMANN, Heidelberg (DE)

(72) Inventors: Frieder LINDER, St. Leon-Rot (DE); Harald SAUER, Heidelberg (DE); Alexander Peter KRÜGER, Heidelberg (DE); Michael Markus HECKMANN, Heidelberg (DE)

(21) Appl. No.: 17/924,741

(22) PCT Filed: Jun. 3, 2020
(86) PCT No.: PCT/EP2020/065290

§ 371 Date: Nov. 11, 2022

(30) Foreign Application Priority Data
May 12, 2020 (DE) 10 2020 112 782.0

(51) Int. Cl. A61L 2/07; A61L 2/26

(52) U.S. Cl. CPC A61L 2/07; A61L 2/26; A61L 2202/122; A61L 2202/14; A61L 2202/24; A61L 2202/26

(57) ABSTRACT

A device for steam disinfecting products includes a treatment chamber. An evaporation pan is arranged in the chamber and heated. A liquid contained in the pan evaporates. A drying air flow is generated by a fan unit and directed onto the evaporation pan via a fan duct. A non-return element is arranged in the fan duct and prevents liquid vapor from getting from the evaporation pan to the fan unit. During an evaporation process, the fan unit is switched off and the fan duct is blocked by a non-return element. During a subsequent drying process, the fan unit is switched on and the non-return element of the fan duct is opened for the drying air flow. During operation of the fan unit, the evaporation pan is heated to a temperature above the specified minimum temperature using a heating device.

### TECHNICAL FIELD

The disclosure relates to a device for steam disinfecting products.

### BACKGROUND

Devices for steam disinfecting products are used for example in hospitals or doctor's practices to disinfect medical equipment. It is also possible to use devices of this kind to disinfect full protective clothing or individual items thereof such as, for example, gloves or face masks and to sterilize same prior to a further re-use.

Devices which have a large-volume treatment chamber which is arranged in a cabinet-like housing are known from practice. Devices of this kind are suitable for steam disinfecting a large number of products. Products arranged in the treatment chamber can be disinfected using hot steam at positive pressure or negative pressure. However, the acquisition and operation of devices of this kind is associated with high costs.

Smaller or low-cost devices for steam disinfection are also known from practice. For example, EP 2 763 705 B1 describes a device for sanitizing and drying products in which an evaporation pan is arranged within a treatment chamber, the device being able to be arranged and operated on a table. A device of this kind requires only a connection to a household power grid and can be set up and put into operation rapidly when needed.

EP 3 409 298 A1 shows and describes a differently designed variant of a device of this kind for sanitizing and drying products, in which the fan unit is arranged not below the evaporation pan but next to the evaporation pan. A drying air flow generated by the fan unit is introduced laterally into the treatment chamber above the evaporation pan and directed onto the heatable evaporation pan. During an operation of this device, the evaporation pan is heated using an electrical heating device and at the same time the fan unit is used to blow an air flow into the treatment chamber. The device described in EP 3 409 298 A1 can be operated only in one single operating mode, in which the evaporation pan is heated and an air flow is also fed, using the fan unit, into the treatment chamber.

### SUMMARY

The disclosure relates to a device for steam disinfecting products. The device comprising a treatment chamber. An evaporation pan which is arranged within the treatment chamber and can be filled with a liquid to be evaporated. A heating device can be used to heat the evaporation pan and a liquid contained therein. A fan unit can be used to generate a drying air flow which is directed onto the evaporation pan via a fan duct connected to the fan unit in the treatment chamber, the fan duct having an orifice opening arranged in the treatment chamber for the out-flowing drying air flow.

The disclosure is based on the realization that the disinfection effect of a liquid vapor which is generated by evaporating a liquid in the heated evaporation pan is noticeably reduced as a result of the simultaneously blown-in air flow, because the air flow suctioned from the ambient air is not separately heated beforehand and is therefore considerably cooler than the liquid vapor in the treatment chamber, which is cooled considerably as a result and loses its disinfecting effect.

The problem addressed by the present disclosure is therefore considered to be that of designing a device of the aforementioned type such that with the simplest possible means, a reliable steam disinfection of products in the treatment chamber can be carried out, and subsequently fastest possible drying of the products treated during the steam disinfection can be carried out. In addition, the operation of the device should be as reliable as possible and it should be possible to carry out an effective steam disinfection of products.

This object is achieved in that a non-return element is arranged in the fan duct, which non-return element can be used to prevent liquid vapor generated by the evaporation pan from getting from the evaporation pan to the fan unit. For a reliable operation of the device, it has been shown to be advantageous if, during a treatment period in which the products are treated with the evaporated liquid vapor, the fan unit is switched off and during the steam disinfection of the products no cool drying air flow is blown into the treatment chamber. With the specified structural arrangement of the fan unit, which feeds the drying air flow to the treatment chamber via the fan duct, wherein the fan duct projects into the treatment chamber above the evaporation pan and has an orifice opening arranged above the evaporation pan, it cannot however be avoided, in the conventional devices, that during the period in which the products are treated with the liquid vapor, some of the liquid vapor enters the fan duct and reaches the fan unit in a direction opposite the drying air flow. The fan unit and more particularly a suction filter provided in the fan unit would then come into contact with the liquid vapor. A liquid condensate precipitating from the cooling liquid vapor would collect not only on inner walls of the fan duct but also in the fan unit and more particularly in the suction filter, and therefore over time a considerable amount of condensate accrues there. This can considerably impair the functioning of the fan unit. Furthermore, it is not possible to rule out that liquid vapor which has already cooled and which penetrates into the fan duct and reaches the fan unit and the suction filter contains germs and possibly bacteria or viruses from the products to be disinfected, which can then build-up in the suction filter and in the fan unit, and therefore when the fan unit is operated during a drying process, the products previously disinfected with the heated liquid vapor are recontaminated.

To reliably prevent the unwanted penetration of the heated liquid vapor into the fan duct and reaches the fan unit, a non-return element is arranged in the fan duct, with which non-return element the unwanted penetration of the liquid vapor can be prevented. During an operation of the fan unit, during which the drying air flow is to be generated and directed through the fan duct into the treatment chamber and onto the evaporation pan, the non-return element is in an open state in which the fan duct is largely unblocked for the drying air flow flowing through. If the fan unit is not in operation, or at least during the treatment period and the evaporation of liquid in the evaporation pan, the non-return element is however in a closed state and reliably closes the fan duct and therefore no liquid vapor can get through the non-return element and reach the fan unit.

According to an embodiment, the non-return element has a blocking flap which is displaceably arranged within the fan duct and in an open position permits a drying air flow generated by the fan unit to flow into the treatment chamber, while in a closed position of the blocking flap, the fan duct is blocked. The blocking flap can be displaceable either in a pivotable manner within the fan duct, or in a linear manner or along a specified trajectory for example along a circular arc segment into and out of the fan duct. The blocking flap can optionally be manually actuatable. It is also conceivable for the blocking flap to be displaceable between the open position and the closed position by means of an actuator unit.

It is optionally provided that the blocking flap is pivotably arranged and can be pivoted into the closed position independently or by spring force actuation. An independently pivotable blocking flap is known from various areas of application such as, for example, in non-return elements in exhaust gas systems. The blocking flap is designed and arranged such that the blocking flap pivots into the closed position through its dead weight and thereby closes the fan duct, while the blocking flap is pivoted into the open position by the flowing drying air flow when there is even a small pressure difference and thus unblocks the fan duct. The blocking flap can optionally also be actuated, or more particularly pulled into the closed position, by a suitable spring unit. As a result, the blocking flap can only be pivoted into the open position when there is a pressure difference specified by the spring unit, and an unwanted opening and unblocking of the fan duct does not take place if the fan unit is not in operation.

According to a particularly advantageous embodiment, the blocking flap can be displaced into the closed position using an automatable actuation unit. The automatable actuation unit can be, for example, a magnetic switch or an electrically operated stepper motor. When an automatable actuation unit is used, the operation of the actuation unit or the displacement of the blocking flap between an open position and a closed position can be specified in an automated manner. This permits steam disinfection and subsequent drying of the steam disinfected products to be carried out in a largely or completely automated manner. Other automatable actuation units are known from practice, which can be used as actuation unit for the blocking flap.

It is also conceivable that an automatable actuation unit is combined with a spring unit or with an independent resetting of the blocking flap. Thus the blocking flap can, for example, be pressed or pulled into an opened open position using a spring, and displaced into the closed position, which closes the fan duct, counter to the spring force or the inherent weight with the aid of a magnetic coil or a magnetic switch.

For an effective steam disinfection, it is advantageous if the products initially to be disinfected with the liquid vapor over the treatment period are then sufficiently dried to prevent the disinfection effect to be impaired by still damp products which are removed from the device after a steam disinfection or by liquid residues still adhering to the products, or to prevent the products having to be subsequently tediously dried outside the treatment chamber. The treatment period needed for a reliable steam disinfection can be of different lengths for different products. For a subsequent drying process to be initiated automatically after the desired treatment period for the product to be disinfected in the individual case the device has a temperature measuring unit with which a temperature range of the evaporation pan can be measured. It has been shown that during a treatment process and the evaporation of liquid in the evaporation pan forced by the heating device, a temperature of the evaporation pan is around 100 degrees Celsius, while after a complete evaporation of the liquid filled in the evaporation pan, this temperature increases rapidly. This temperature increase can be measured using the temperature measuring unit and be used as the trigger for a drying process after the period of treatment with the evaporating liquid. The heating unit must merely be suitable for heating the evaporation pan significantly over 100 degrees Celsius. This is readily possible in the case of many commercially available heating devices.

Using a temperature measuring unit makes it possible to specify the desired steam disinfection treatment period by filling the evaporation pan with a corresponding amount of liquid. The treatment period then corresponds to the duration needed for a complete evaporation of the amount of liquid filled in the evaporation pan. As soon as the liquid has completely evaporated and a temperature value of the evaporation pan heated by the heating device rises significantly over 100 degrees Celsius, the steam disinfection of the products can be ended and the subsequent drying process initiated in that the fan unit is switched on and a drying air flow flowing into the treatment chamber is generated.

The temperature measuring unit can, for example, be a temperature-sensitive sensor with a microcontroller which evaluates the measurement signals of the temperature-sensitive sensor. The temperature measuring unit can have a thermal element and enable a comparatively precise temperature measurement which is exact to less than a few degrees within a temperature range of between around 0 degrees Celsius and 150 degrees Celsius.

According to an embodiment, it is provided that the device has a first bimetal switch connected in a thermally conductive manner to the evaporation pan, with which bimetal switch an operation of the heating device of the evaporation pan can be interrupted as soon as a temperature of the evaporation pan rises above a specified maximum value. The first bimetal switch can be arranged for example on an underside of the evaporation pan and is triggered when a temperature value of the evaporation pan rises above a maximum value specified by the embodiment of the first bimetal switch, at which maximum value the bimetal switch deforms and thus interrupts a electric circuit of the heating device, so that the heating device is switched off and a heating of the evaporation pan rises above the maximum value specified by the first bimetal switch. The first bimetal switch has only two different switching states, and therefore no complex measurement signal processing is needed for evaluating the measurement signals of the first bimetal switch. A suitable first bimetal switch is commercially available and can be installed and used at low effort to control the heating device. The first bimetal switch can be arranged in a electric circuit for the heating device in such a manner that when the evaporation pan is heated above a maximum value specified by the first bimetal switch, the first bimetal switch changes its switching state and interrupts this electric circuit, so that the heating device is switched off by the first bimetal switch and the evaporation pan cools down. As soon as the temperature of the evaporation pan falls below a minimum value likewise specified by the first bimetal switch, the first bimetal switch deforms again and the electric circuit for the heating device is closed again, and therefore the heating device heats the evaporation pan again.

The advantage of using a first bimetal switch to interrupt the electric circuit of the heating device over other control options is that depending on the temperature, the electric circuit for the heating device is interrupted mechanically, which excludes errors which cannot be completely excluded for example in an evaluation of sensors with a microcontroller circuit.

In view of highest possible operational safety and the desired prevention of an unwanted overheating of the heating device or the evaporation pan heated therewith, it is proposed that the device has a second bimetal switch which redundantly to the first bimetal switch is connected in a thermally conductive manner to the evaporation pan. The second bimetal switch can have the same trigger properties as the first bimetal switch and be arranged in series with the first bimetal switch in the electric circuit of the heating device and therefore when the evaporation pan is heated above the specified maximum temperature value, both bimetal switches are triggered and after the first trigger of one of the two bimetal switches the electric circuit is already interrupted. The two bimetal switches therefore effect a redundant monitoring and prevention of the unwanted exceeding of the maximum temperature.

It is also possible that, in addition to the two bimetal switches, a separate safety unit is provided to avoid an overheating. Thus for example a fusible-wire fuse can be arranged in the electric circuit of the heating device and interrupt the electric circuit if the current flow exceeds a maximum value specified by the fusible-wire fuse. Other safety units are also known and commercially available which can be used to effect a one-time and permanent interruption of the electric circuit independently of a bimetal switch which is usually designed reversibly.

In view of providing operation of the device that is as automated as possible, it is optionally provided that the device has a control unit with which the heating device and the fan unit can be controlled. The control unit preferably has an evaluation unit with which a measurement signal of a sensor unit can be evaluated and, when an evaporation process has come to an end, operation of the fan unit can be started. In principle, the operation of the heating device and the fan unit can each be specified manually and activated for example by actuating corresponding switches, for example. Since the period for treating products to ensure a reliable steam disinfection should usually last several minutes, however, and the subsequent drying of the products regularly also lasts several minutes and up to half an hour or longer, a manual actuation of the heating device and of the fan unit, and thus an active intervention by a user, would be required at intervals of a few minutes, which reduces the comfort of using a device of this kind. With a control unit set up and operated in a suitable manner, in particular in conjunction with the evaluation unit, the products can first be treated with heated liquid vapor and a drying process can then be initiated in an automated manner. The duration for treating the products with the heated liquid vapor can be specified in a simple manner either using a separate timer or by means of the fill quantity of the liquid which is filled in the evaporation pan before the start of the evaporation process. The evaluation unit can be used to automatically detect the end of the evaporation process and set the device to the drying mode. For this purpose, a change in the temperature of the evaporation pan occurring after the complete evaporation of the liquid can be monitored using a temperature measuring unit, for example, and based on a sharp temperature increase, such as that which occurs after the evaporation of the liquid, the end of the evaporation process can be determined. It is also possible to use a suitable sensor to detect the flow of current through the electric circuit of the heating device and to regard the first triggering of a bimetal switch, which interrupts this electric circuit and suddenly stops the current flow, as the end of the evaporation process. The current flow can preferably be detected in a contactless manner and carried out for example with the aid of a measuring coil or a Hall sensor. A contactless or galvanically isolated detection of a change in the current flow indicating the end of the evaporation process has the advantage that the evaluation unit is not connected in an electrically conductive manner to an electric circuit operated with 230 V or power electronics components for the operation of the device and more particularly of the heating device. The duration of the drying process can in turn be specified by a manually operable timer or a corresponding electronic controller.

To accelerate the drying processes, it is provided to advantage that the drying air flow, which supports and accelerates the drying process, is additionally heated. For this purpose, the evaporation pan can be heated further during the drying process and held at a high temperature level. The drying air flow flowing through the fan duct and out of the orifice opening in the direction of the evaporation pan then brushes along the heated evaporation pan, absorbs thermal energy and heats up. A separate heating device for the drying air flow generated using the fan unit is therefore not necessary. This enables a particularly low-cost production of the device.

The disclosure also relates to a method for steam disinfecting products in a treatment chamber, the products in the treatment chamber being exposed, over a treatment period, to a heated liquid vapor which is generated in an evaporation pan which can be heated using a heating device, the products then being dried using a drying air flow which is introduced via the fan duct into the treatment chamber by a fan unit. The method described for example in EP 3 409 298 A1 requires the fan unit to also be operated during the period during which the products are treated with the heated liquid vapor and thus a drying air flow to be blown into the treatment chamber. It has however been shown that this can impair a reliable steam disinfection, because as a result of the drying air flow blown into the treatment chamber, the temperature of the heated liquid vapor is considerably reduced.

A separate heating of the drying air flow, which is generated by the fan unit and blown through the fan duct into the treatment chamber, would be associated with a considerable additional design effort. The method described in EP 3 409 298 A1 should therefore be changed if possible to enable a steam disinfection of products and subsequent drying of the products that is as simple and cost-effective as possible.

[symbol] , this object is achieved in that during the steam disinfection treatment period the fan unit is switched off and the fan duct is blocked by a non-return element, after the treatment period has elapsed the fan unit is switched on and the non-return element opens the fan duct for the drying air flow, and during operation of the fan unit the evaporation pan is heated to above a specified minimum temperature using a heating device. The fan unit is expediently switched off during the period in which the products are treated with the heated liquid vapor, and therefore no cool drying air flow is introduced into the treatment chamber and the heated liquid vapor can have a temperature of almost 100 degrees Celsius over the treatment period. This leads to a particularly reliable steam disinfection of the products in the treatment chamber.

Since the fan unit is switched off during the treatment period, a portion of the heated liquid vapor could penetrate into the fan duct and reach the switched-off fan unit, as a result of which said unit could be impaired or contaminated by condensate collecting there. To prevent this, the invention proposes blocking the fan duct with the aid of a non-return element during the period of treatment with the heated liquid vapor, so that no liquid vapor can get through the fan duct and reach the fan unit via the non-return element.

To heat the drying air flow generated by the fan duct during a subsequent drying process, without the need for an additional separate heating device for the drying air flow, the evaporation pan is heated using the heating device during operation of the fan unit and is kept continuously above a specified minimum temperature. The drying air flow blown in through the fan duct heats up on contact with the heated evaporation pan, and the temperature in the treatment chamber therefore increases and the drying process is accelerated.

According to a particularly advantageous embodiment of the method, the drying air flow is directed out of an orifice opening of the fan duct in the direction of the evaporation pan and is then distributed in the treatment chamber. Since the drying air flow blown into the treatment chamber is oriented to the evaporation pan, an intensive contact of the drying air flow with the heated evaporation pan is forced and the drying air flow is heated more effectively because it brushes along a surface of the evaporation pan.

In view of an advantageous automation of the method sequence, it is optionally provided that a specified maximum value of the temperature of the evaporation pan is optionally measured using a temperature measuring unit and thereby the end of the treatment period is determined. It is also conceivable that the treatment period can be specified manually using a timer. Such a specification of the treatment period can be carried out in that, when switching on a device used for steam disinfection, a user selects a product to be disinfected from a specified number of products or a product set which is to be disinfected during the subsequent treatment process with the heated liquid vapor.

It is expediently optionally provided that after a specifiable drying period has elapsed, the fan unit and the heating device are switched off. The method can thereby be carried out and completed without a further intervention being required by the user. Energy can be saved by switching off the fan unit and the heating device after a specifiable drying period. If individual disinfected products are not sufficiently dry, a further drying process can be easily initiated and an additional drying period specified.

In a particularly advantageous manner, an above-described device is used to carry out the method.

### BRIEF DESCRIPTION OF THE DRAWINGS

Below, an exemplary embodiment of the inventive concept is described in more detail by way of example and is shown in the drawing.

FIG. 1 is a sectional view of a device for steam disinfecting products;

FIG. 2 is a flow chart for a schematically illustrated method sequence of a method for steam disinfecting products,

FIG. 3 a temperature curve over time of an evaporation pan, heated using a heating device, of the device shown in FIG. 1, and

FIG. 4 a temperature curve over time of air in a treatment chamber of the device shown in FIG. 1, the air first being enriched with a heated liquid vapor and then a drying air flow being applied to the air.

### DETAILED DESCRIPTION

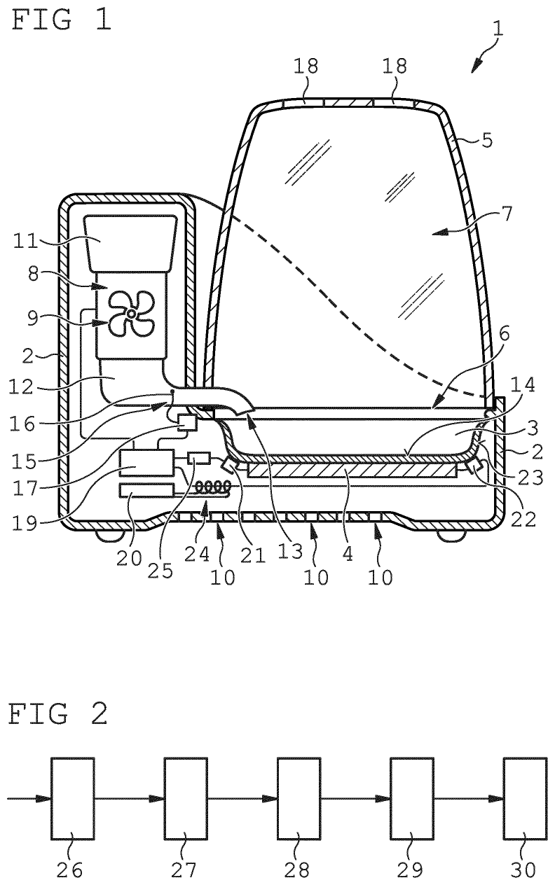

A device 1 schematically shown in FIG. 1 for steam disinfecting products, not shown in FIG. 1, has a housing 2, in which an evaporation pan 3 is arranged. The evaporation pan 3 is connected in a heat-conducting manner to a heating device 4 arranged below the evaporation pan 3. The heating device 4 can be used to heat the evaporation pan 3. The heating device 4 is designed and can be operated such that the evaporation pan 3 can be heated to a temperature of more than 140 degrees Celsius. A liquid can be filled in the evaporation pan 3, which liquid is gradually evaporated during the heating of the evaporation pan 3 by the heating device 4 and thus forms a rising heated liquid vapor. A housing cover 5 made of a transparent material can be placed on an opening 6 of the housing 2, said opening unblocking the evaporation pan, and above the evaporation pan 3 forms a treatment chamber 7 largely closed by the housing cover 5. The products to be disinfected can be arranged in this treatment chamber 7.

Laterally next to the evaporation pan 3 in the housing 2, a fan unit 8 having a merely schematically indicated fan 9 is arranged. During an operation of the fan unit 8, an air flow is suctioned through a suction region 10 of the housing, which suction region is located in the base region of the housing 2. As a result, it is possible to avoid, for example during a filling process of the evaporation pan 3 with an amount of liquid, liquid being accidentally spilled over a suction region and penetrating into the fan unit 8. The ambient air suctioned through the suction region 10 in the base of the housing 2 is then guided through a suction filter 11. The suction filter 11 can be, for example, a particle filter or a suspended matter filter with which viruses and bacteria can also optionally be filtered out of the drying air flow suctioned in from outside the housing 2. The suction filter 11 is supported in an exchangeable manner in the housing 2, and therefore the suction filter 11 can be exchanged when needed.

The drying air flow suctioned in and thereby generated by the fan unit 8 is then blown laterally into the treatment chamber 7 through a fan duct 12. To this end, an opening of the fan duct 12 opens directly into the evaporation pan 3, the fan duct projecting into the treatment chamber 7 above the evaporation pan 3. As a result of the course of the fan duct 12 in the region of the orifice opening, the drying air flow generated using the fan unit 8 is directed onto the evaporation pan 3 and after exiting the orifice opening 13 flows along a surface 14 of the evaporation pan 3 and can thereby heat up.

In the fan duct 12, a merely schematically indicated non-return element 15 is arranged which has a blocking flap 16 which is mounted in a pivoting manner in the fan duct 12. In a closed position, the blocking flap 16 closes the fan duct 12 and prevents a liquid vapor generated by an evaporation of liquid in the evaporation pan 3 from being able to get into the fan duct 12 and reaching the fan unit 8.

The blocking flap 16 can be actuated with the aid of a magnetic switch 17 and can be displaced from a closed position to an open position or back. In an open position, the blocking flap 16 unblocks the fan duct 12 and therefore the drying air flow generated using the fan unit 8 can be blown through the fan duct 12 onto the evaporation pan 3 and introduced into the treatment chamber 7. The housing cover 5 has venting slots 18 out of which the air in the treatment chamber 7 can flow during an operation of the device 1 so that a pressure equalization between the treatment chamber 7 and the ambient air is possible.

The device 1 has a control unit 19 and an evaluation unit 20 connected thereto in a signal-transmitting manner. The control unit 19 can be used to control the operation of the fan unit 8 and the operation of the heating device 4. The control unit 19 can also be used to control and actuate the magnetic switch 19 for actuating the non-return element 15. Furthermore, a first bimetal switch 21 and a second bimetal switch 22 are connected to the control unit 19, which bimetal switches are arranged in series with the heating device 4 in an electric circuit which supplies the heating device 4 with electrical energy during a heating process. The first bimetal switch 21 and the second bimetal switch 22 are each fixed in a heat-conducting manner to an outer face 23 of the evaporation pan 3 facing away from the treatment chamber 7. The first bimetal switch 21 and the second bimetal switch 22 are dimensioned such that both bimetal switches 21, 22 transition from a first switching state to a second switching state when the outer side 23 of the evaporation pan 3 is heated to over 140 degrees Celsius. The arrangement of the two bimetal switches 21, 22 in series leads to a redundant switching-off of the electric circuit for the heating device as soon as the temperature of the outer side 23 of the evaporation pan 3 rises above a specified maximum temperature. When the outer side 23 of the evaporation pan 3 cools to below 80° Celsius, the two bimetal switches 21, 22 change their switching state and the electric circuit is closed again. With the aid of the evaluation unit 20, the current flow through the electric circuit of the heating device 4 or a sudden drop in the current flow when at least one of the bimetal switches 21, 22 is triggered can be identified by means of a sensor device 24. Said sudden drop in the current flow in the event of a first-time triggering of one of the two bimetal switches 21, 22 can be regarded as the end of the evaporation process and can initiate a subsequent drying process. Using an additional safety device 25, for example a fusible-wire fuse, a further monitoring of the heating device 4 can be carried out in addition to the two bimetal switches 21, 22 and an unwanted overheating of the heating device 4 or of the evaporation pan 3 can be prevented.

FIG. 2 schematically shows an example of a method sequence for a method for steam disinfecting products. The method can be carried out using the device 1 shown in FIG. 1.

In a preparatory step 26, a specified amount of a liquid must first be filled in the evaporation pan 3. The liquid can be distilled water, for example. The products to be treated are arranged in the treatment chamber 7 above the evaporation pan 3 and the treatment chamber 7 is then closed.

In a subsequent switch-on step 27, the device 1 is switched on for example by actuation of a switch, or if the device 1 is already switched on, an automated treatment process is initiated. First, a treatment of the products arranged in the treatment chamber 7 for steam disinfection is initiated.

During a treatment step 28, the heating device 4 is used to heat the evaporation pan 3 and the liquid filled therein gradually evaporates. The heated liquid vapor rises and surrounds the products arranged in the treatment chamber 7, as a result of which said products are steam disinfected. The duration of the treatment step, or the treatment period, is specified by the amount of liquid filled in the evaporation pan 3, which evaporates during the treatment step. In many cases, it is expedient for the treatment of the products with the heated liquid vapor to be carried out over a period of several minutes, for example 5 minutes. During the treatment step 28, the fan duct 12 is closed by the non-return element 15, so that no heated liquid vapor can get through the fan duct 12 and reach the fan unit 8 and suction filter 11.

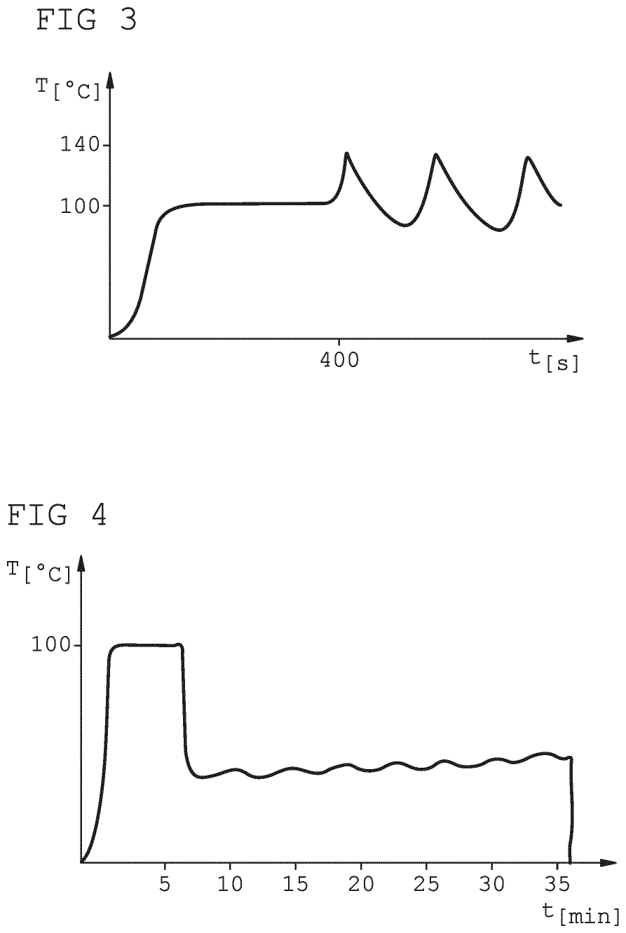

During the operation of the heating device 4 during the treatment step 28, the evaporation pan 3 initially heats up comparatively quickly to a temperature value of around 100 degrees Celsius, as shown in the schematically represented temperature curve in FIG. 3. A measured temperature of the evaporation pan 3 over a time of several minutes is shown here. During the evaporation of the liquid, the temperature of the evaporation pan 3 does not rise much over 100° Celsius. As soon as the liquid has completely evaporated, however, the temperature of the evaporation pan 3 continues to rise rapidly. As soon as the temperature of the evaporation pan 3 rises to a temperature above 140 degrees Celsius, the first bimetal switch 21 and the second bimetal switch 22, which are arranged redundantly in series in the electric circuit, are triggered and thereby interrupt the electric circuit for the heating device 4. Consequently, the evaporation pan 3 gradually cools down, and therefore its temperature falls to a value below 100 degrees Celsius. This prevents the heating device 4 and the evaporation pan 3 from overheating. As soon as the temperature of the evaporation pan 3 falls below a minimum temperature of around 90 degrees Celsius, the two bimetal switches 21, 22 change their switching state and close the electric circuit of the heating device 4 again, causing the heating device 4 to be switched on again and put into operation. As a result, the temperature of the evaporation pan 3 rises rapidly again. This repeated switching on and switching off of the heating device 4 can be carried out over a long period of time in order to support a drying of the products after the treatment step 28. The temperature values given in this exemplary embodiment are merely examples, and it is therefore possible to have a maximum temperature of more than or less than 140 degrees Celsius or a minimum temperature of more than or less than 90 degrees Celsius.

As a result of the first-time rise in the temperature of the evaporation pan 3 up to the maximum temperature of 140 degrees Celsius, the treatment step 28 is automatically ended and a subsequent drying step 29 initiated. In the drying step 29, the non-return element 15 is first placed into an open state, wherein the blocking flap 16 is pivoted by the magnetic switch 17 into an open position in which it unblocks the fan duct 12. Using the fan unit 8, a drying air flow is generated which is suctioned through the suction filter 11 and is blown through the fan duct 12 onto the surface 14 of the evaporation pan 3. As a result, the drying air flow, which was not previously separately heated, heats up and is then distributed within the treatment chamber 7 and dries the products located therein. A drying process of this kind can last around 20 to 30 minutes. The duration of the drying process, or of drying step 29, can be specified by means of a timer to practically any value and depending on the products to be dried and the fill state of the treatment chamber 7. During the drying step 29, the temperature of the evaporation pan 3 is held in a temperature range specified by the minimum temperature and the maximum temperature and thereby the air in the treatment chamber 7 is heated and the drying process accelerated. Because the drying air flow brushes along the surface 14 of the heated evaporation pan 3, the drying air flow, which is subsequently distributed in the treatment chamber 7, heats up. As soon as a specified time period has elapsed, the heating device 4 and the fan unit 8 are switched off in a switch-off step 30.

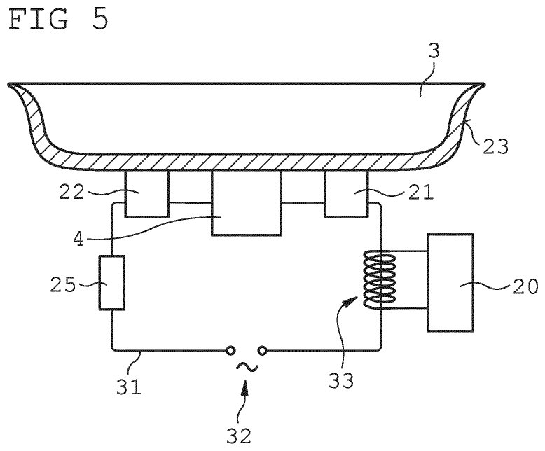

FIG. 4 schematically shows the time curve of a temperature of the air within the treatment chamber 7, said temperature being measured by a separate sensor. During the treatment period or during the evaporation process within the treatment step 28, the liquid filled in the evaporation pan 3 evaporates and the liquid vapor heated to around 100 degrees Celsius fills the treatment chamber 7. At the end of the evaporation process and thus at the end of the treatment step 28, the drying air flow heated by the evaporation pan 3, which continues to be heated, is distributed in the treatment chamber 7 during the subsequent drying step 29. The temperature over time of the air in the treatment chamber 7 follows the temperature over time of the evaporation plate 3 heated by the heating device 3, wherein during the drying step 27, the temperature in the treatment chamber 7 initially falls to around 45 degrees Celsius and then gradually rises to 60 degrees Celsius and more during the drying process. The drying step 29 lasts around 30 minutes.

At the specified end of the drying period, or of drying step 29, the device 1 is automatically switched off in the switch-off step 30. The steam disinfected and subsequently dried products can then be removed from the treatment chamber 7.

FIG. 5 shows merely schematically an electric circuit 31 for electrically supplying the heating device 4 of the evaporation pan 3. The electric circuit 31 is supplied, by means of an energy supply 32, not shown in more detail, of a household power grid for example with 115 V or 230 V alternating voltage. Two bimetal switches 21 and 22 connected in series with the heating device 4 are fixed to an outer side 23 of the evaporation pan 3. A safety unit 25 also connected in series constitutes an additional thermal fuse. The sensor unit 24 can be used to measure the current flow through the electric circuit 31 in a contactless manner. For this purpose, the sensor unit 24 has a magnetic coil 33, which can be used to detect the magnetic field generated by the current flow in the electric circuit 31.

1.-14. (canceled)

15. A device (1) for steam disinfecting products, comprising:
a treatment chamber (7);
an evaporation pan (3) arranged within the treatment chamber (7) suitable to be filled with a liquid to be evaporated;
a heating device (4) for heating the evaporation pan (3) and evaporating a liquid located therein;
a fan unit (8) for generating a drying air flow;
a fan duct (12) for directing the drying air flow onto the evaporation pan (3) in the treatment chamber (7), the fan duct being connected to the fan unit (8);
an orifice opening (13) of the fan duct (12) arranged in the treatment chamber (7) for the outflowing drying air flow; and
a non-return element (15) arranged in the fan duct (12) to prevent vapor generated by the evaporation pan (3) from traveling from the evaporation pan (3) to the fan unit (8).

16. The device (1) according to claim 15,
wherein the non-return element (15) has a blocking flap (16) which is displaceably arranged within the fan duct (12) and which,
in an open position, permits the drying air flow generated by the fan unit (8) to flow into the treatment chamber (7), and which,
in a closed position, blocks the fan duct (12).

17. The device (1) according to claim 16,
wherein the blocking flap (16) is pivotably arranged and can be pivoted into the closed position independently or by spring force actuation.

18. The device (1) according to claim 16,
wherein the blocking flap (16) can be displaced into the closed position using an automatable actuation unit.

19. The device (1) according to claim 15, further comprising
a temperature measuring unit for measuring a temperature of the evaporation pan (3).

20. The device (1) according to claim 15, further comprising
a first bimetal switch (21) connected in a heat-conducting manner to the evaporation pan (3),
wherein the first bimetal switch (21) interrupts an operation of the heating device (4) of the evaporation pan (3) as soon as a temperature of the evaporation pan (3) rises above a specified maximum value.

21. The device (1) according to claim 20, further comprising
a second bimetal switch (22) which redundantly to the first bimetal switch (21) is connected in a heat-conducting manner to the evaporation pan (3).

22. The device (1) according to claim 15, further comprising
a control unit (19) for controlling the heating device (4) and the fan unit (8).

23. The device (1) according to claim 22,
wherein the control unit (19) comprises an evaluation unit (20) for evaluating a measurement signal of a sensor unit (25) and for activating the fan unit (8) when an evaporation process has come to an end.

24. A method for steam disinfecting products in a treatment chamber (7), comprising:
exposing, over a treatment period, the products in the treatment chamber (7) to a vapor of a heated liquid which is generated in an evaporation pan (3) that is heated using a heating device (4);
drying the products using a drying air flow which is introduced via a fan duct (12) into the treatment chamber (7) by a fan unit (8);
switching off the fan unit (8) and blocking the fan duct (12) by a non-return element (15) during the treatment period;
switching on the fan unit (8) and causing the non-return element (15) to open the fan duct (12) for a drying air flow after the treatment period has elapsed; and
heating, during operation of the fan unit (8), the evaporation pan (3) to above a specified minimum temperature using the heating device (4).

25. The method according to claim 24,
wherein the drying air flow is directed out of an orifice opening (13) of the fan duct (12) in the direction of the evaporation pan (3) and is then distributed in the treatment chamber (7).

26. The method according to claim 24, further comprising
measuring a specified maximum value of the temperature of the evaporation pan (3) with a sensor unit (25) and thereby determining an end of the treatment period.

27. The method according to claim 24, further comprising
switching off the fan unit (8) and the heating device (4) after a specifiable drying period has elapsed.
